(19) United States
(12) Patent Application Publication
(10) Pub. No.: US 2018/0218793 A1
(43) Pub. Date: Aug. 2, 2018

(54) PHYSICIAN EXAMINATION SCHEDULING SYSTEM AND PROCESSES TO SELF-REPORT SYMPTOMS FOR AN EXAMINATION

(71) Applicant: Michael Edwin Hebrard, Piedmont, CA (US)

(72) Inventor: Michael Edwin Hebrard, Piedmont, CA (US)

(21) Appl. No.: 15/878,651

(22) Filed: Jan. 24, 2018

Related U.S. Application Data
(60) Provisional application No. 62/451,585, filed on Jan. 27, 2017.

(51) Int. Cl. G16H 50/50; G16H 80/00; G06Q 10/10; G06F 17/30

(52) U.S. Cl. CPC G16H 50/50; G16H 80/00; G06F 17/3087; G06F 17/30604; G06Q 10/1095

(57) ABSTRACT

An in-person physician examination scheduling system and processes for pairing mobile devices to initiate patient communication with a nearby physician and for using 3D human anatomical models to self-report symptoms to the physician for an in-person examination are disclosed. The in-person physician examination scheduling system provides two-way communication between patients with urgent medical needs and physicians who are available to facilitate in an in-person examination. The in-person physician examination scheduling system utilizes comprehensive medical algorithms with the assistance of a 3D human anatomical model to improve diagnostic accuracy and timely treatments during physician-patient evaluations. The in-person physician examination scheduling system records geospatial data, enabling the analysis of real-time medical trends by demographic criteria and geographical regions. The in-person physician examination scheduling system provides visual annotations of patients' symptoms using the 3D human anatomical model to describe and illustrate symptoms.

### CROSS-REFERENCE TO RELATED APPLICATIONS

This application claims priority to U.S. Provisional Patent Application No. 62/451,585 filed Jan. 27, 2017. The entire contents and disclosures of this patent application are incorporated herein by reference in its entirety.

### BACKGROUND

### Field of the Invention

Embodiments of the invention described in this specification relate generally to doctor scheduling systems, and more particularly, to an in-person physician examination scheduling system and processes for pairing mobile devices to initiate patient communication with a nearby physician and for using 3D human anatomical models to self-report symptoms to the physician for an in-person examination.

### Background of the Invention

Limited access to urgent medical care creates delays in treatment, increased diagnostic and treatment errors. The result increases disease severity, elevated cost, provider “burnout”, and a host of other problems. Many such problems could be alleviated or eliminated with better use of existing technology. For instance, none of the existing systems includes mobile applications in which there is duality in the app platform allowing a patient (using one mobile device) to initiate contact with an available physician (using another mobile device).

### SUMMARY

According to a first broad aspect, the present disclosure provides a process to self-report symptoms to a physician for an in-person examination, comprising: transmitting a patient mobile application to a patient mobile device associated with a patient who has a medical issue and is seeking in-person examination and diagnosis of the medical issue to be treated, wherein when the patient mobile application is installed on the patient mobile device the patient is able to seek in-person examination and treatment from an available nearby physician in a pool of nearby physicians; transmitting a physician mobile application to a physician mobile device associated with a physician who is available to examine patients nearby a physical location of the physician, wherein when the physician mobile application is installed on the physician mobile device, an identify and qualifications of the physician are verified, and upon receiving physician approval to receive nearby requests for appointments by patients, the physician is listed among a pool of physicians in an area that is nearby the physical location of the patient; receiving, from the patient mobile application running on the patient mobile device, answers to a series of questions paired with one or more 3D human anatomical model(s) to gather initial patient data; receiving, from the patient mobile application running on the patient mobile device, an opt-in request to locate a nearby physician and schedule an in-person appointment; receiving, from the patient mobile application running on the patient mobile device, an approval to participate in and pay for an in-person examination according to the scheduling of the in-person appointment; transmitting a request to schedule the in-person appointment to the physician mobile application running on the physician mobile device; receiving an acceptance, from the physician mobile application running on the physician mobile device, to schedule the in-person appointment to examine the patient; utilizing, by the physician mobile application running on the physician mobile device, a data platform and predictive diagnoses to complete an in-person examination of the patient; and prescribing a treatment plan for the patient based on the completed in-person examination of the patient by the physician.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

Having thus described the invention in general terms, reference is now made to the accompanying drawings, which are not necessarily drawn to scale, and which show different views of different example embodiments.

FIG. 1 is a schematic illustration showing a scheduling system and processes to self-report symptoms for an examination according to one embodiment of the present disclosure.

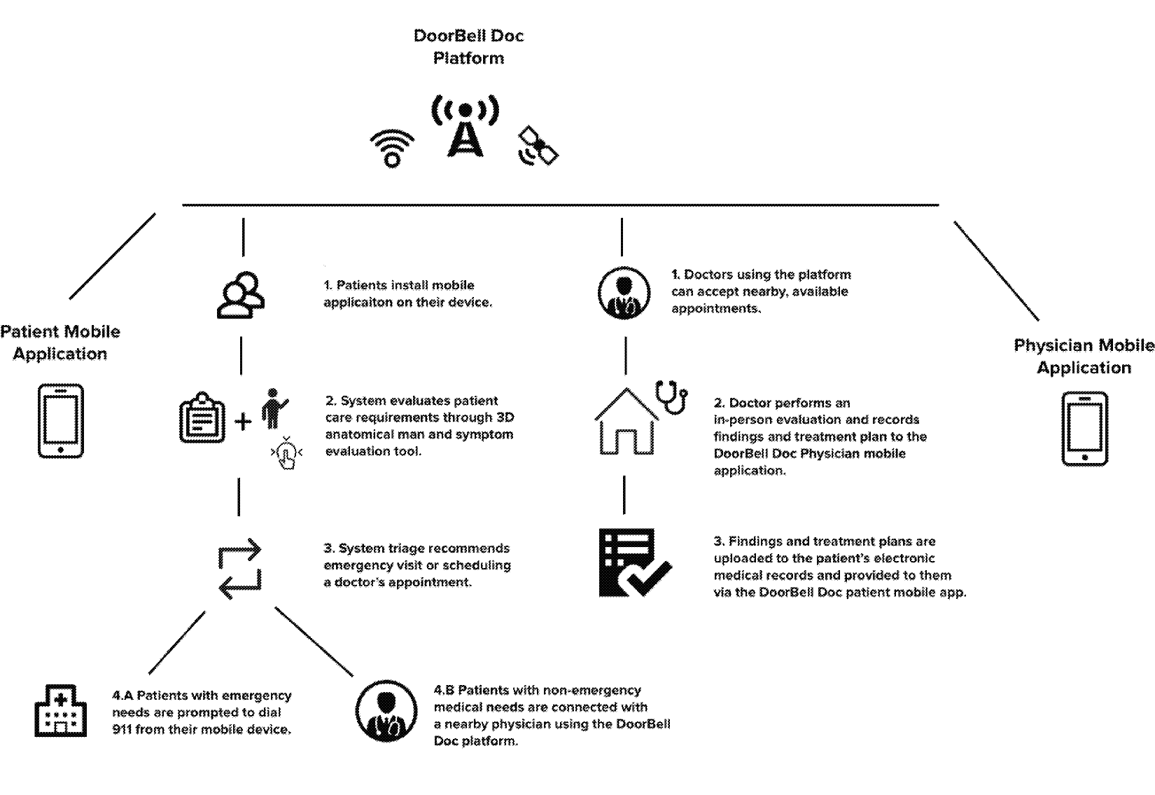

### DETAILED DESCRIPTION

### Definitions

Where the definition of terms departs from the commonly used meaning of the term, applicant intends to utilize the definitions provided below, unless specifically indicated.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

For purposes of the present disclosure, the term “comprising”, the term “having”, the term “including,” and variations of these words are intended to be open-ended and mean that there may be additional elements other than the listed elements.

For purposes of the present disclosure, directional terms such as “top,” “bottom,” “upper,” “lower,” “above,” “below,” “left,” “right,” “horizontal,” “vertical,” “up,” “down,” etc., are used merely for convenience in describing the various embodiments of the present disclosure. The embodiments may be oriented in various ways. For example, the diagrams, apparatuses, etc., shown in the drawing figures may be flipped over, rotated by 90° in any direction, reversed, etc.

For purposes of the present disclosure, a value or property is “based” on a particular value, property, the satisfaction of a condition, or other factor, if that value is derived by performing a mathematical calculation or logical decision using that value, property or other factor.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

For purposes of the present disclosure, the term “associated” with respect to data refers to data that are associated or linked to each other. For example, data relating the identity of an individual (identity data) wearing an integrated sensor module may be associated with the motion data for the individual obtained from an accelerometer or, optionally, from a gyroscope or, optionally, from the amplitude of the power signal from an energy harvester.

For the purposes of the present disclosure, the term “Bluetoote” refers to a wireless technology standard for exchanging data over short distances (using short-wavelength radio transmissions in the ISM band from 2400-2480 MHz) from fixed and mobile devices, creating personal area networks (PANs) with high levels of security. Created by telecom vendor Ericsson in 1994, it was originally conceived as a wireless alternative to RS-232 data cables. It can connect several devices, overcoming problems of synchronization. Bluetooth® is managed by the Bluetooth® Special Interest Group, which has more than 18,000 member companies in the areas of telecommunication, computing, networking, and consumer electronics. Bluetooth® was standardized as IEEE 802.15.1, but the standard is no longer maintained. The SIG oversees the development of the specification, manages the qualification program, and protects the trademarks. To be marketed as a Bluetooth® device, it must be qualified to standards defined by the SIG. A network of patents is required to implement the technology and is licensed only for those qualifying devices.

For the purposes of the present disclosure, the term “cloud computing” is synonymous with computing performed by computers that are located remotely and accessed via the Internet (the “Cloud”). It is a style of computing where the computing resources are provided “as a service”, allowing users to access technology-enabled services “in the cloud” without knowledge of, expertise with, or control over the technology infrastructure that supports them. According to the IEEE Computer Society it “is a paradigm in which information is permanently stored in servers on the Internet and cached temporarily on clients that include desktops, entertainment centers, table computers, notebooks, wall computers, handhelds, etc.” Cloud computing is a general concept that incorporates virtualized storage, computing and web services and, often, software as a service (SaaS), where the common theme is reliance on the Internet for satisfying the computing needs of the users. For example, Google Apps provides common business applications online that are accessed from a web browser, while the software and data are stored on the servers. Some successful cloud architectures may have little or no established infrastructure or billing systems whatsoever including Peer-to-peer networks like BitTorrent and Skype and volunteer computing like SETI@home. The majority of cloud computing infrastructure currently consists of reliable services delivered through next-generation data centers that are built on computer and storage virtualization technologies. The services may be accessible anywhere in the world, with the Cloud appearing as a single point of access for all the computing needs of data consumers. Commercial offerings may need to meet the quality of service requirements of customers and may offer service level agreements. Open standards and open source software are also critical to the growth of cloud computing. As customers generally do not own the infrastructure, they are merely accessing or renting, they may forego capital expenditure and consume resources as a service, paying instead for what they use. Many cloud computing offerings have adopted the utility computing model which is analogous to how traditional utilities like electricity are consumed, while others are billed on a subscription basis. By sharing “perishable and intangible” computing power between multiple tenants, utilization rates may be improved (as servers are not left idle) which can reduce costs significantly while increasing the speed of application development. A side effect of this approach is that “computer capacity rises dramatically” as customers may not have to engineer for peak loads. Adoption has been enabled by “increased high-speed bandwidth” which makes it possible to receive the same response times from centralized infrastructure at other sites.

For purposes of the present disclosure, the term “computer” refers to any type of computer or other device that implements software including an individual computer such as a personal computer, laptop computer, tablet computer, mainframe computer, mini-computer, etc. A computer also refers to electronic devices such as an electronic scientific instrument such as a spectrometer, a smartphone, an eBook reader, a cell phone, a television, a handheld electronic game console, a videogame console, a compressed audio or video player such as an MP3 player, a Blu-ray player, a DVD player, etc. In addition, the term “computer” refers to any type of network of computers, such as a network of computers in a business, a computer bank, the Cloud, the Internet, etc. Various processes of the present disclosure may be carried out using a computer. Various functions of the present disclosure may be performed by one or more computers.

For the purposes of the present disclosure, the term “computer hardware” and the term “hardware” refer to the digital circuitry and physical devices of a computer system, as opposed to computer software, which is stored on a hardware device such as a hard disk. Most computer hardware is not seen by normal users, because it is embedded within a variety of every day systems, such as in automobiles, microwave ovens, electrocardiograph machines, compact disc players, and video games, among many others. A typical personal computer consists of a case or chassis in a tower shape (desktop) and the following parts: motherboard, CPU, RAM, firmware, internal buses (PIC, PCI-E, USB, HyperTransport, CSI, AGP, VLB), external bus controllers (parallel port, serial port, USB, Firewire, SCSI. PS/2, ISA, EISA, MCA), power supply, case control with cooling fan, storage controllers (CD-ROM, DVD, DVD-ROM, DVD Writer, DVD RAM Drive, Blu-ray, BD-ROM, BD Writer, floppy disk, USB Flash, tape drives, SATA, SAS), video controller, sound card, network controllers (modem, NIC), and peripherals, including mice, keyboards, pointing devices, gaming devices, scanner, webcam, audio devices, printers, monitors, etc.

For the purposes of the present disclosure, the term “computer network” refers to a group of interconnected computers. Networks may be classified according to a wide variety of characteristics. The most common types of computer networks in order of scale include: Personal Area Network (PAN), Local Area Network (LAN), Campus Area Network (CAN), Metropolitan Area Network (MAN), Wide Area Network (WAN), Global Area Network (GAN), Internetwork (intranet, extranet, Internet), and various types of wireless networks. All networks are made up of basic hardware building blocks to interconnect network nodes, such as Network Interface Cards (NICs), Bridges, Hubs, Switches, and Routers. In addition, some method of connecting these building blocks is required, usually in the form of galvanic cable (most commonly category 5 cable). Less common are microwave links (as in IEEE 802.11) or optical cable (“optical fiber”).

For the purposes of the present disclosure, the term “computer software” and the term “software” refers to one or more computer programs, procedures and documentation that perform some tasks on a computer system. The term includes application software such as word processors which perform productive tasks for users, system software such as operating systems, which interface with hardware to provide the necessary services for application software, and middleware which controls and co-ordinates distributed systems. Software may include websites, programs, video games, etc. that are coded by programming languages like C, C++, Java, etc. Computer software is usually regarded as anything but hardware, meaning the “hard” are the parts that are tangible (able to hold) while the “soft” part is the intangible objects inside the computer. Computer software is so called to distinguish it from computer hardware, which encompasses the physical interconnections and devices required to store and execute (or run) the software. At the lowest level, software consists of a machine language specific to an individual processor. A machine language consists of groups of binary values signifying processor instructions which change the state of the computer from its preceding state.

For the purposes of the present disclosure, the term “computer system” refers to any type of computer system that implements software including an individual computer such as a personal computer, mainframe computer, mini-computer, etc. In addition, computer system refers to any type of network of computers, such as a network of computers in a business, the Internet, personal data assistant (PDA), devices such as a cell phone, a television, a videogame console, a compressed audio or video player such as an MP3 player, a DVD player, a microwave oven, etc. A personal computer is one type of computer system that typically includes the following components: a case or chassis in a tower shape (desktop) and the following parts: motherboard, CPU, RAM, firmware, internal buses (PIC, PCI-E, USB, HyperTransport, CSI, AGP, VLB), external bus controllers (parallel port, serial port, USB, Firewire, SCSI. PS/2, ISA, EISA, MCA), power supply, case control with cooling fan, storage controllers (CD-ROM, DVD, DVD-ROM, DVD Writer, DVD RAM Drive, Blu-ray, BD-ROM, BD Writer, floppy disk, USB Flash, tape drives, SATA, SAS), video controller, sound card, network controllers (modem, NIC), and peripherals, including mice, keyboards, pointing devices, gaming devices, scanner, webcam, audio devices, printers, monitors, etc.

For the purposes of the present disclosure, the term “data” means the reinterpretable representation of information in a formalized manner suitable for communication, interpretation, or processing. Although one type of common type data is a computer file, data may also be streaming data, a web service, etc. The term “data” is used to refer to one or more pieces of data.

For the purposes of the present disclosure, the term “database” or “data record” refers to a structured collection of records or data that is stored in a computer system. The structure is achieved by organizing the data according to a database model. The model in most common use today is the relational model. Other models such as the hierarchical model and the network model use a more explicit representation of relationships (see below for explanation of the various database models). A computer database relies upon software to organize the storage of data. This software is known as a database management system (DBMS). Database management systems are categorized according to the database model that they support. The model tends to determine the query languages that are available to access the database. A great deal of the internal engineering of a DBMS, however, is independent of the data model, and is concerned with managing factors such as performance, concurrency, integrity, and recovery from hardware failures. In these areas there are large differences between products.

For the purposes of the present disclosure, the term “database management system (DBMS)” represents computer software designed for the purpose of managing databases based on a variety of data models. A DBMS is a set of software programs that controls the organization, storage, management, and retrieval of data in a database. DBMS are categorized according to their data structures or types. It is a set of prewritten programs that are used to store, update and retrieve a Database.

For the purposes of the present disclosure, the term “data storage medium” or “data storage device” refers to any medium or media on which a data may be stored for use by a computer system. Examples of data storage media include floppy disks, Zip™ disks, CD-ROM, CD-R, CD-RW, DVD, DVD-R, memory sticks, flash memory, hard disks, solid state disks, optical disks, etc. Two or more data storage media acting similarly to a single data storage medium may be referred to as a “data storage medium” for the purposes of the present disclosure. A data storage medium may be part of a computer.

For purposes of the present disclosure, the term “hardware and/or software” refers to functions that may be performed by digital software, digital hardware, or a combination of both digital hardware and digital software. Various features of the present disclosure may be performed by hardware and/or software.

For purposes of the present disclosure, the term “individual” refers to an individual mammal, such as a human being.

For purposes of the present disclosure, the term “HIPAA” refers to Health Insurance Portability and Accountability Act of 1996. HIPAA is United States legislation that provides data privacy and security provisions for safeguarding medical information. Sections 261 through 264 of HIPAA require the Secretary of Health and Human Services (HSS) to publicize standards for the electronic exchange, privacy and security of health information.

For the purposes of the present disclosure, the term “Internet” is a global system of interconnected computer networks that interchange data by packet switching using the standardized Internet Protocol Suite (TCP/IP). It is a “network of networks” that consists of millions of private and public, academic, business, and government networks of local to global scope that are linked by copper wires, fiber-optic cables, wireless connections, and other technologies. The Internet carries various information resources and services, such as electronic mail, online chat, file transfer and file sharing, online gaming, and the inter-linked hypertext documents and other resources of the World Wide Web (WWW).

For the purposes of the present disclosure, the term “Internet protocol (IP)” refers to a protocol used for communicating data across a packet-switched internetwork using the Internet Protocol Suite (TCP/IP). IP is the primary protocol in the Internet Layer of the Internet Protocol Suite and has the task of delivering datagrams (packets) from the source host to the destination host solely based on its address. For this purpose the Internet Protocol defines addressing methods and structures for datagram encapsulation. The first major version of addressing structure, now referred to as Internet Protocol Version 4 (Ipv4) is still the dominant protocol of the Internet, although the successor, Internet Protocol Version 6 (Ipv6) is actively deployed world-wide. In one embodiment, an EGI-SOA of the present disclosure may be specifically designed to seamlessly implement both of these protocols.

For the purposes of the present disclosure, the term “intranet” refers to a set of networks, using the Internet Protocol and IP-based tools such as web browsers and file transfer applications that are under the control of a single administrative entity. That administrative entity closes the intranet to all but specific, authorized users. Most commonly, an intranet is the internal network of an organization. A large intranet will typically have at least one web server to provide users with organizational information. Intranets may or may not have connections to the Internet. If connected to the Internet, the intranet is normally protected from being accessed from the Internet without proper authorization. The Internet is not considered to be a part of the intranet.

For the purposes of the present disclosure, the term “local area network (LAN)” refers to a network covering a small geographic area, like a home, office, or building. Current LANs are most likely to be based on Ethernet technology. The cables to the servers are typically on Cat 5e enhanced cable, which will support IEEE 802.3 at 1 Gbit/s. A wireless LAN may exist using a different IEEE protocol, 802.11b, 802.11g or possibly 802.11n. The defining characteristics of LANs, in contrast to WANs (wide area networks), include their higher data transfer rates, smaller geographic range, and lack of a need for leased telecommunication lines. Current Ethernet or other IEEE 802.3 LAN technologies operate at speeds up to 10 Gbit/s.

For the purposes of the current disclosure, the term “low powered wireless network” refers to an ultra-low powered wireless network between sensor nodes and a centralized device. The ultra-low power is needed by devices that need to operate for extended periods of time from small batteries energy scavenging technology. Examples of low powered wireless networks are ANT, ANT+, Bluetooth Low Energy (BLE), ZigBee and WiFi.

For purposes of the present disclosure, the term “machine-readable medium” refers to any tangible or non-transitory medium that is capable of storing, encoding or carrying instructions for execution by the machine and that cause the machine to perform any one or more of the methodologies of the present disclosure, or that is capable of storing, encoding or carrying data structures utilized by or associated with such instructions. The term “machine-readable medium” includes, but is limited to, solid-state memories, and optical and magnetic media. Specific examples of machine-readable media include non-volatile memory, including by way of example, semiconductor memory devices, e.g., EPROM, EEPROM, and flash memory devices; magnetic disks such as internal hard disks and removable disks; magneto-optical disks; and CD-ROM and DVD-ROM disks. The term “machine-readable medium” may include a single medium or multiple media (e.g., a centralized or distributed database, and/or associated caches and servers) that store the one or more instructions or data structures.

For the purposes of the present disclosure the term “mobile ad hoc network” is a self-configuring infrastructureless network of mobile devices connected by wireless. Ad hoc is Latin and means “for this purpose”. Each device in a mobile ad hoc network is free to move independently in any direction, and will therefore change its links to other devices frequently. Each must forward traffic unrelated to its own use, and therefore be a router. The primary challenge in building a mobile ad hoc network is equipping each device to continuously maintain the information required to properly route traffic. Such networks may operate by themselves or may be connected to the larger Internet. Mobile ad hoc networks are a kind of wireless ad hoc networks that usually has a routable networking environment on top of a Link Layer ad hoc network. The growths of laptops and wireless networks have made mobile ad hoc networks a popular research topic since the mid-1990s. Many academic papers evaluate protocols and their abilities, assuming varying degrees of mobility within a bounded space, usually with all nodes within a few hops of each other. Different protocols are then evaluated based on measure such as the packet drop rate, the overhead introduced by the routing protocol, end-to-end packet delays, network throughput etc.

For the purposes of the present disclosure, the term “network hub” refers to an electronic device that contains multiple ports. When a packet arrives at one port, it is copied to all the ports of the hub for transmission. When the packets are copied, the destination address in the frame does not change to a broadcast address. It does this in a rudimentary way, it simply copies the data to all of the Nodes connected to the hub. This term is also known as hub. The term “Ethernet hub,” “active hub,” “network hub,” “repeater hub,” “multiport repeater” or “hub” may also refer to a device for connecting multiple Ethernet devices together and making them act as a single network segment. It has multiple input/output (I/O) ports, in which a signal introduced at the input of any port appears at the output of every port except the original incoming. A hub works at the physical layer (layer 1) of the OSI model. The device is a form of multiport repeater. Repeater hubs also participate in collision detection, forwarding a jam signal to all ports if it detects a collision.

For purposes of the present disclosure, the term “non-transient storage medium” refers to a storage medium that is non-transitory, tangible and computer readable. Non-transient storage medium may refer generally to any durable medium known in the art upon which data can be stored and later retrieved by data processing circuitry operably coupled with the medium. A non-limiting non-exclusive list of exemplary non-transitory data storage media may include magnetic data storage media (e.g., hard disc, data tape, etc.), solid state semiconductor data storage media (e.g., SDRAM, flash memory, ROM, etc.), and optical data storage media (e.g., compact optical disc, DVD, etc.).

For purposes of the present disclosure, the term “processor” refers to a device that performs the basic operations in a computer. A microprocessor is one example of a processor.

For the purposes of the present disclosure, the term “random-access memory (RAM)” refers to a type of computer data storage. Today it takes the form of integrated circuits that allow the stored data to be accessed in any order, i.e. at random. The word random thus refers to the fact that any piece of data can be returned in a constant time, regardless of its physical location and whether or not it is related to the previous piece of data. This contrasts with storage mechanisms such as tapes, magnetic discs and optical discs, which rely on the physical movement of the recording medium or a reading head. In these devices, the movement takes longer than the data transfer, and the retrieval time varies depending on the physical location of the next item. The word RAM is mostly associated with volatile types of memory (such as DRAM memory modules), where the information is lost after the power is switched off. However, many other types of memory are RAM as well, including most types of ROM and a kind of flash memory called NOR-Flash.

For the purposes of the present disclosure, the term “read-only memory (ROM)” refers to a class of storage media used in computers and other electronic devices. Because data stored in ROM cannot be modified (at least not very quickly or easily), it is mainly used to distribute firmware (software that is very closely tied to specific hardware, and unlikely to require frequent updates). In its strictest sense, ROM refers only to mask ROM (the oldest type of solid state ROM), which is fabricated with the desired data permanently stored in it, and thus can never be modified. However, more modern types such as EPROM and flash EEPROM can be erased and re-programmed multiple times; they are still described as “read-only memory” because the reprogramming process is generally infrequent, comparatively slow, and often does not permit random access writes to individual memory locations.

For the purposes of the present disclosure, the term “real-time processing” refers to a processing system designed to handle workloads whose state is constantly changing. Real-time processing means that a transaction is processed fast enough for the result to come back and be acted on as transaction events are generated. In the context of a database, real-time databases are databases that are capable of yielding reliable responses in real-time. For the purposes of the present disclosure, the term “router” refers to a networking device that forwards data packets between networks using headers and forwarding tables to determine the best path to forward the packets. Routers work at the network layer of the TCP/IP model or layer 3 of the OSI model. Routers also provide interconnectivity between like and unlike media devices. A router is connected to at least two networks, commonly two LANs or WANs or a LAN and its ISP' s network.

For the purposes of the present disclosure, the term “server” refers to a system (software and suitable computer hardware) that responds to requests across a computer network to provide, or help to provide, a network service. Servers can be run on a dedicated computer, which is also often referred to as “the server,” but many networked computers are capable of hosting servers. In many cases, a computer can provide several services and have several servers running. Servers may operate within a client-server architecture and may comprise computer programs running to serve the requests of other programs—the clients. Thus, the server may perform some task on behalf of clients. The clients typically connect to the server through the network but may run on the same computer. In the context of Internet Protocol (IP) networking, a server is a program that operates as a socket listener. Servers often provide essential services across a network, either to private users inside a large organization or to public users via the Internet. Typical computing servers are database server, file server, mail server, print server, web server, gaming server, application server, or some other kind of server. Numerous systems use this client/server networking model including Web sites and email services. An alternative model, peer-to-peer networking may enable all computers to act as either a server or client as needed.

For the purposes of the present disclosure, the term “solid-state electronics” refers to those circuits or devices built entirely from solid materials and in which the electrons, or other charge carriers, are confined entirely within the solid material. The term is often used to contrast with the earlier technologies of vacuum and gas-discharge tube devices and it is also conventional to exclude electro-mechanical devices (relays, switches, hard drives and other devices with moving parts) from the term solid state. While solid-state can include crystalline, polycrystalline and amorphous solids and refer to electrical conductors, insulators and semiconductors, the building material is most often a crystalline semiconductor. Common solid-state devices include transistors, microprocessor chips, and RAM. A specialized type of RAM called flash RAM is used in flash drives and more recently, solid state drives to replace mechanically rotating magnetic disc hard drives. More recently, the integrated circuit (IC), the light-emitting diode (LED), and the liquid-crystal display (LCD) have evolved as further examples of solid-state devices. In a solid-state component, the current is confined to solid elements and compounds engineered specifically to switch and amplify it.

For purposes of the present disclosure, the term “storage medium” refers to any form of storage that may be used to store bits of information. Examples of storage media include both volatile and non-volatile memories such as MRRAM, MRRAM, ERAM, flash memory, RFID tags, floppy disks, Zip™ disks, CD-ROM, CD-R, CD-RW, DVD, DVD-R, flash memory, hard disks, optical disks, etc. Two or more storage media acting similarly to a single data storage medium may be referred to as a “storage medium” for the purposes of the present disclosure. A storage medium may be part of a computer.

For the purposes of the present disclosure, the term “transmission control protocol (TCP)” refers to one of the core protocols of the Internet Protocol Suite. TCP is so central that the entire suite is often referred to as “TCP/IP.” Whereas IP handles lower-level transmissions from computer to computer as a message makes its way across the Internet, TCP operates at a higher level, concerned only with the two end systems, for example a Web browser and a Web server. In particular, TCP provides reliable, ordered delivery of a stream of bytes from one program on one computer to another program on another computer. Besides the Web, other common applications of TCP include e-mail and file transfer. Among its management tasks, TCP controls message size, the rate at which messages are exchanged, and network traffic congestion.

For the purposes of the present disclosure, the term “time” refers to a component of a measuring system used to sequence events, to compare the durations of events and the intervals between them, and to quantify the motions of objects. Time is considered one of the few fundamental quantities and is used to define quantities such as velocity. An operational definition of time, wherein one says that observing a certain number of repetitions of one or another standard cyclical event (such as the passage of a free-swinging pendulum) constitutes one standard unit such as the second, has a high utility value in the conduct of both advanced experiments and everyday affairs of life. Temporal measurement has occupied scientists and technologists, and was a prime motivation in navigation and astronomy. Periodic events and periodic motion have long served as standards for units of time. Examples include the apparent motion of the sun across the sky, the phases of the moon, the swing of a pendulum, and the beat of a heart. Currently, the international unit of time, the second, is defined in terms of radiation emitted by cesium atoms.

For the purposes of the present disclosure, the term “timestamp” refers to a sequence of characters, denoting the date and/or time at which a certain event occurred. This data is usually presented in a consistent format, allowing for easy comparison of two different records and tracking progress over time; the practice of recording timestamps in a consistent manner along with the actual data is called timestamping. Timestamps are typically used for logging events, in which case each event in a log is marked with a timestamp. In file systems, timestamp may mean the stored date/time of creation or modification of a file. The International Organization for Standardization (ISO) has defined ISO 8601 which standardizes timestamps.

For the purposes of the present disclosure, the term “visual display device” or “visual display apparatus” includes any type of visual display device or apparatus such as a CRT monitor, LCD screen, LEDs, a projected display, a printer for printing out an image such as a picture and/or text, etc. A visual display device may be a part of another device such as a computer monitor, television, projector, telephone, cell phone, smartphone, laptop computer, tablet computer, handheld music and/or video player, personal data assistant (PDA), handheld game player, head mounted display, a heads-up display (HUD), a global positioning system (GPS) receiver, automotive navigation system, dashboard, watch, microwave oven, electronic organ, automatic teller machine (ATM) etc.

For the purposes of the present disclosure, the term “web service” refers to the term defined by the W3C as “a software system designed to support interoperable machine-to-machine interaction over a network”. Web services are frequently just web APIs that can be accessed over a network, such as the Internet, and executed on a remote system hosting the requested services. The W3C Web service definition encompasses many different systems, but in common usage the term refers to clients and servers that communicate using XML messages that follow the SOAP standard. In such systems, there is often machine-readable description of the operations offered by the service written in the Web Services Description Language (WSDL). The latter is not a requirement of a SOAP endpoint, but it is a prerequisite for automated client-side code generation in many Java and .NET SOAP frameworks. Some industry organizations, such as the WS-I, mandate both SOAP and WSDL in their definition of a Web service. More recently, RESTful Web services have been used to better integrate with HTTP compared to SOAP-based services. They do not require XML messages or WSDL service-API definitions.

For the purposes of the present disclosure, the term “wide area network (WAN)” refers to a data communications network that covers a relatively broad geographic area (i.e. one city to another and one country to another country) and that often uses transmission facilities provided by common carriers, such as telephone companies. WAN technologies generally function at the lower three layers of the OSI reference model: the physical layer, the data link layer, and the network layer.

### Description

In the following detailed description of the invention, numerous details, examples, and embodiments of the invention are described. However, it will be clear and apparent to one skilled in the art that the invention is not limited to the embodiments set forth and that the invention can be adapted for any of several applications.

While the disclosure is open to various modifications and alternative forms, specific embodiments thereof have been shown by way of example in the drawings and will be described in detail below. It should be understood, however that it is not intended to limit the disclosure to the particular forms disclosed, but on the contrary, the disclosure is to cover all modifications, equivalents, and alternatives falling within the spirit and the scope of the disclosure.

Existing systems fail to utilize comprehensive medical algorithms with the assistance of a three-dimensional (hereinafter abbreviated as “3D”) human anatomical model, which details common symptoms with the corresponding organ systems. This relates also to the problem of failure to provide visual annotations of patients' symptoms. But a 3D human anatomical model approach could help reduce diagnostic and treatment delays and errors.

Another problem of the existing systems is the overall failure to document real time geospatial trends with illness affecting specific demographics, delaying timely, comprehensive medical care and increasing the spread of disease outbreaks.

Therefore, what is needed is a way to provide two-way communication between patients with urgent medical needs and doctors who are available to facilitate in an in-person examination, while using comprehensive medical algorithms with the assistance of a 3D 2 human anatomical model which increases diagnostic accuracy and timely treatments during doctor-patient evaluations, as well as recording geospatial data, enabling the analysis of real time medical trends by demographic criteria and geographical regions, and providing visual annotations of patients' symptoms using the 3D human anatomical model.

Some embodiments of the invention include a novel in-person physician examination scheduling system and novel processes for pairing mobile devices to initiate patient communication with a nearby physician and for using 3D human anatomical models to self-report symptoms to the physician for an in-person examination. In some embodiments, the in-person physician examination scheduling system connects a patient with self-reported symptoms to a nearby physician for an in-person examination. In some embodiments, the processes for pairing mobile devices to initiate patient communication with a nearby physician and for using 3D human anatomical models to self-report symptoms to the physician for an in-person examination involves pairing mobile devices of the patient and the physician via a mobile app that implements the processes and visually outputs the 3D human anatomical model to allow the patient to self-report health symptoms.

Some embodiments provide an in-person physician examination scheduling system. In some embodiments, the in-person physician examination scheduling system connects a patient with self-reported symptoms to a nearby physician for an in-person examination.

Some embodiments provide a process for pairing mobile devices to initiate patient communication with a nearby physician from a pool of available nearby physicians. In some embodiments, the process for pairing mobile devices to initiate patient communication with a nearby physician includes linking mobile devices of the patient and the physician via dual mobile apps (the mobile app being installed on each mobile device).

Some embodiments provide a process for using 3D human anatomical models to self-report symptoms to the physician for an in-person examination. In some embodiments, the process for using 3D human anatomical models is implemented as a mobile app that visually outputs the 3D human anatomical model to allow the patient to self-report health symptoms.

As stated above, limited access to urgent medical care creates delays in treatment, increased diagnostic and treatment errors, which collectively results in increased disease severity, elevated cost, provider “burnout”, and a host of other problems. Technology-based visual and communication systems would present a new era in tackling these problems. However, none of the existing systems in the medical field utilize such technology. Another problem of the existing systems is the overall failure to document real time geospatial trends with illness affecting specific demographics, delaying timely, comprehensive medical care and increasing the spread of disease outbreaks. Embodiments of the invention described in this specification solve such problems by an in-person physician examination scheduling system and processes for pairing mobile devices to initiate patient communication with a nearby physician and for using 3D human anatomical models to self-report symptoms to the physician for an in-person examination which uses dual software apps that connect patients with urgent medical needs and treating physicians in a digital mobile environment reducing emergency room visits, and that provides timely access to quality and comprehensive medical care. The result will improve diagnostic accuracy and treatment outcomes while saving on healthcare cost.

Embodiments of the in-person physician examination scheduling system and the processes for pairing mobile devices to initiate patient communication with a nearby physician and for using 3D human anatomical models to self-report symptoms to the physician for an in-person examination described in this specification differ from and improve upon currently existing options. In particular, some embodiments of the in-person physician examination scheduling system and the processes for pairing mobile devices to initiate patient communication with a nearby physician and for using 3D human anatomical models to self-report symptoms to the physician for an in-person examination differ by use of dual mobile apps to connect patients with physicians and facilitate the medical examination process through digital and in-person examinations. The unique use of a 3D anatomical man paired with predictive diagnosis algorithms facilitates timely access to comprehensive urgent medical care.

In addition, the in-person physician examination scheduling system and the processes for pairing mobile devices to initiate patient communication with a nearby physician and for using 3D human anatomical models to self-report symptoms to the physician for an in-person examination of some embodiments improve upon the currently existing options because the existing systems lack the technology to facilitate a patient-centered healthcare delivery system in a digital mobile environment. In contrast, the in-person physician examination scheduling system and the processes for pairing mobile devices to initiate patient communication with a nearby physician and for using 3D human anatomical models to self-report symptoms to the physician for an in-person examination provide two-way communication between patients with urgent medical needs and physicians who are available to facilitate in an in-person examination. In some embodiments, the use of comprehensive medical algorithms with the assistance of a 3D anatomical human increases diagnostic accuracy and timely treatments during physician-patient evaluations. Additionally, the in-person physician examination scheduling system and the processes for pairing mobile devices to initiate patient communication with a nearby physician and for using 3D human anatomical models to self-report symptoms to the physician for an in-person examination record geospatial data, enabling the analysis of real time medical trends by demographic criteria and geographical regions. In particular, the in-person physician examination scheduling system and the processes for pairing mobile devices to initiate patient communication with a nearby physician and for using 3D human anatomical models to self-report symptoms to the physician for an in-person examination of some embodiments include features that are implemented as software (mobile apps) which provide visual annotations of patients' symptoms using a 3D anatomical model to describe and illustrate symptoms. The benefits are magnified because the in-person physician examination scheduling system and the processes for pairing mobile devices to initiate patient communication with a nearby physician and for using 3D human anatomical models to self-report symptoms to the physician for an in-person examination of some embodiments incorporate a linked duality between the mobile apps on the mobile devices of physician and patient, thereby allowing the patient to initiate contact with the physician (e.g., a nearby physician who is currently available) and utilize one or more 3D human anatomical models (specifically, 3D computer generated graphics, or CGI, of human anatomical models) to visually identify symptoms corresponding to human organ systems or other human biological systems.

In some embodiments, as the steps in the in-person physician examination scheduling process are completed, the in-person physician examination scheduling system can gather and anonymously identify unique trends related to the data consumed. For example:

1. The in-person physician examination scheduling system and the processes for pairing mobile devices to initiate patient communication with a nearby physician and for using 3D human anatomical models to self-report symptoms to the physician for an in-person examination can identify common trends ranging from small neighborhood communities to more global magnitudes to identify outbreaks of symptoms, helping to track the potential spread of disease.

2. The in-person physician examination scheduling system and the processes for pairing mobile devices to initiate patient communication with a nearby physician and for using 3D human anatomical models to self-report symptoms to the physician for an in-person examination can identify keywords in a physician's treatment plan, which allows the invention to identify over or under prescription of various medical drugs or treatments.

3. The in-person physician examination scheduling system and the processes for pairing mobile devices to initiate patient communication with a nearby physician and for using 3D human anatomical models to self-report symptoms to the physician for an in-person examination can utilize machine learning to train our predictive diagnostic treatment algorithms to improve the quality of healthcare outcomes.

4. The in-person physician examination scheduling system and the processes for pairing mobile devices to initiate patient communication with a nearby physician and for using 3D human anatomical models to self-report symptoms to the physician for an in-person examination can utilize a 3D human anatomical model (a CGI representation of a 3D human anatomical model on a computing device screen) to help patients explain their symptoms and improve the provider's understanding of the patient's complaints and guide the provider's evaluation diagnosis and treatment plan.

The in-person physician examination scheduling system and the processes for pairing mobile devices to initiate patient communication with a nearby physician and for using 3D human anatomical models to self-report symptoms to the physician for an in-person examination of the present disclosure may be comprised of the following elements and steps. This list of possible constituent elements and steps is intended to be exemplary only and it is not intended that this list be used to limit the in-person physician examination scheduling system and the processes for pairing mobile devices to initiate patient communication with a nearby physician and for using 3D human anatomical models to self-report symptoms to the physician for an in-person examination of the present application to just these elements and steps. Persons having ordinary skill in the art relevant to the present disclosure may understand there to be equivalent elements or steps that may be substituted within the present disclosure without changing the essential function or operation of the in-person physician examination scheduling system and the processes for pairing mobile devices to initiate patient communication with a nearby physician and for using 3D human anatomical models to self-report symptoms to the physician for an in-person examination.

1. Software (e.g., mobile apps) installed on mobile devices for both patients and providers.

2. Patient provides description of symptoms utilizing 3D human anatomical models and medical algorithms

3. Patient input will triage their condition for urgent vs. emergency medical care.

4. Emergency triage will prompt the patient to call 911.

5. Non-emergency triage will prompt the patient to request a house-call medical evaluation.

6. The nearest available network provider will respond to the evaluation request.

7. Patient and provider meet for in-home face-to-face evaluation.

8. The physician reviews the patient's chief symptoms and electronic medical records.

9. Provider uses a medical algorithm to document pertinent clinical exam findings.

10. Provider is given a list of probable diagnoses based on their documented evaluation.

11. Treatment plan will be generated based on the selected diagnosis.

12. Results of the evaluation will be uploaded to the patient's electronic medical records.

The various elements and steps of the in-person physician examination scheduling system and the processes for pairing mobile devices to initiate patient communication with a nearby physician and for using 3D human anatomical models to self-report symptoms to the physician for an in-person examination of the present disclosure may be related in the following exemplary fashion. It is not intended to limit the scope or nature of the relationships between the various elements and the following examples are presented as illustrative examples only.

1-2: The installation of the software (mobile apps) on mobile devices which have a screen capable of rendering CGI representations of 3D human anatomical model(s) enables patients to view the 3D human anatomical model(s) and select symptoms which are captured by the mobile app (and device) and then transmitted to a system server (e.g., a cloud server that hosts a cloud application service) for subsequent transmission to an evaluating physician or pool of nearby physicians available to treat the patient in person (traditionally referred to as a house call).

2-3: Determine the severity of the patient's symptoms for a specific treatment.

3-4-5: Based on the results of the triage, patients are prompted for an emergency call or house-call evaluation.

6-7: Use of geographical coordinates allows our system to match a patient with a nearby network provider.

7-8: Provider access to the patient's electronic health record enhances comprehensive medical evaluation and treatment.

8-11: Based on the provider's documented clinical findings, a diagnosis and treatment plan will be generated and uploaded to the patient's electronic health records.

The in-person physician examination scheduling system and the processes for pairing mobile devices to initiate patient communication with a nearby physician and for using 3D human anatomical models to self-report symptoms to the physician for an in-person examination of the present disclosure generally work by collection of patient symptoms coupled with organ systems and clinical exam findings to produce a medically reasonable diagnosis and treatment plan in a digital mobile environment. Visual annotations of patients' symptoms using a 3D human anatomical model to describe and illustrate symptoms. The platform also stores symptom complexes correlating with specific organ systems and clinical findings, which provide a selection of probable diagnoses and treatments.

To make the in-person physician examination scheduling system and the processes for pairing mobile devices to initiate patient communication with a nearby physician and for using 3D human anatomical models to self-report symptoms to the physician for an in-person examination of the present disclosure, one would need to develop technology to break down barriers that delay or deny access to good comprehensive urgent medical care by using a digital mobile medical app that connects patients and providers. In doing so, one may develop modules, software, and/or systems including:

1. Development of a dual digital mobile app capable of connecting patient and providers while interfacing digital medical algorithms capturing patients' symptom complexes correlating with organ systems, provider utilization of a medical algorithm to document pertinent clinical exam findings that generate a selection of probable diagnosis and treatment protocols while allowing access to the patient's electronic medical records for comprehensive assessment and appropriate treatment.

2. Development of one or more interactive 3D human anatomical models that give patients the ability to precisely communicate their symptoms in a visual format, thereby improving patient provider communication and subsequently proper treatment.

3. Creation of a secure digital mobile app that's simple to use for both patients and providers utilizing a series of one click responses to document patient symptoms and clinical exam findings.

4. Creation of a data platform capable of collecting and analyzing clinical information to assess the prevalence and incidence of diseases which would reduce epidemic disease spread and provide timely precise treatment. The data platform may be created in a way suitable to work with the interactive 3D human anatomical model(s) to enable patients to visually explain their ailment(s). The data platform may be comprehensive in scope sufficient to include a relational data platform of human organ systems and other human biological systems, symptoms, examination findings, and potential diagnoses.

In some embodiments, the in-person physician examination scheduling system includes machine learning modules and/or algorithms to train one or more predictive diagnostic treatment algorithms to improve healthcare treatment outcomes via utilization of the dual mobile applications, which work in tandem to initiate contact between patient and physician, identify symptoms, transfer electronic medical records information between patient, patient's primary medical provider, and treating physician in a secure, HIPAA compliant and patient authorized manner, and identify a physical location of the patient so that the treating physician can travel to the location to examine and/or treat the patient in person.

In some embodiments, the in-person physician examination scheduling system is associated with a mobile application platform to which the dual mobile apps are published. In some embodiments, the in-person physician examination scheduling system is HIPAA compliant. While HIPAA compliance is required by law in some regions (i.e., the United States), it is noted here that HIPAA compliance is not unique or required for the in-person physician examination scheduling system to operate.

In some embodiments, the in-person physician examination scheduling process includes the following steps (as implemented by the software):

1. Gathering initial patient data through a series of one-click responses and interaction with a 3D human anatomical model.

2. Pairing mobile device of the patient with a mobile device of a nearby, available physician based on geospatial points gathered from both the mobile device of the patient and the mobile device of the physician.

3. Enabling the physician to make an in-person evaluation that results in the presentation of predictive diagnoses through a data platform and internal algorithms of the in-person physician examination scheduling system.

4. Allowing the patient to access their subsequent treatment plan and have that plan uploaded to their electronic health records.

Some deviation from the above steps is possible without affecting the overall function and operation of the in-person physician examination scheduling process. The following features exemplify the flexibility of the in-person physician examination scheduling process, which when implemented as software, allows a patient to use a 3D human anatomical model to self-report health symptoms.

1. Predictive diagnosis step during which a physician or other qualified healthcare professional makes a preliminary in-person diagnosis, after which the process presents one or more 3D human anatomical model(s) that visually represent the affected systems and organs.

2. The 3D human anatomical model(s) are rendered as 3D computer graphics imagery (“CGI”) visuals which can be adapted in any of several forms, shapes, views or vantage points, etc., such that each 3D human anatomical model is visually output on the patient's mobile device screen to represent alternative models with varying systems and interactivity (e.g., different organ systems or other biological systems).

3. While the system is based on the concept of dual mobile apps running on mobile computing devices of patient and physician, one could remove the physician component, presenting patients merely with suggested diagnoses.

For a patient to use the in-person physician examination scheduling system and the processes for pairing mobile devices to initiate patient communication with a nearby physician and for using 3D human anatomical models to self-report symptoms to the physician for an in-person examination of the present disclosure, the patient may employ the following exemplary steps:

1. Install the patient mobile application.

2. Answer a series of one-click questions paired with one or more 3D human anatomical model(s) to gather initial patient data.

3. Opt-in to locate a nearby physician and schedule an appointment.

4. Participate in and pay for an at-home (in-person) examination.

5. View current and past treatment plans.

For a physician to use the in-person physician examination scheduling system and the processes for pairing mobile devices to initiate patient communication with a nearby physician and for using 3D human anatomical models to self-report symptoms to the physician for an in-person examination of the present disclosure, the physician may employ the following exemplary steps:

1. Install the physician mobile application.

2. Verify their identity and qualifications.

3. Accept nearby requests for appointments by patients.

4. Utilize the system's data platform and predictive diagnoses to complete their in-person examination of the patient.

5. Prescribe a treatment plan for the patient.

By using the in-person physician examination scheduling system and the processes for pairing mobile devices to initiate patient communication with a nearby physician and for using 3D human anatomical models to self-report symptoms to the physician for an in-person examination in this way, patients are able to obtain timely access to comprehensive urgent medical care, while physicians are able to provide their medical services at nearby locations to patients in need.

In addition to the dual mobile application/device functionality that enables a patient to find a nearby physician, the in-person physician examination scheduling system and processes of some embodiments can gather and anonymously identify unique trends related to the data consumed. Examples of other features utilized by and trends identifiable to the in-person physician examination scheduling system include, without limitation, the following:

1. Identification of common trends ranging from small neighborhood communities to more global magnitudes to identify outbreaks of symptoms, helping to track the potential spread of disease.

2. Identification of keywords in a physician's treatment plan, which allows for identification of over or under prescription of various medical drugs or treatments.

3. Utilization of machine learning to train the predictive diagnostic treatment algorithms to improve the quality of healthcare outcomes.

4. Utilization of any of several varying 3D human anatomical models to help patients explain their symptoms and improve the physician or provider's understanding of the patient's complaints and/or to guide the physician or provider's evaluation, diagnosis, and treatment plan.

Embodiments of the present disclosure provide an artificial intelligence (AI) module to provide machine learning capability. Exemplary examples are provided in the disclosed software application as follows: embodiments of the present disclosure may provide ongoing clinical data input of prescribed concierge doctors to generate the most mathematically probable diagnosis and treatment options. Based on the physicians' documented clinical findings, the disclosed system will automatically produce potential diagnosis and treatment options that can be selected and administered by the physicians. Through machine learning, artificial intelligence (AI), the physicians' clinical data trains the software system how to recognize and respond with the most accurate predictive diagnosis and treatment. The treatment protocol serves in an advisory capacity only, and can be overridden by the physician, should they deem necessary, and in the best interest of the patient. Full and final treatment decisions rest with the expertise of the doctors. At the same time, the disclosed system provides predictive diagnosis and treatment plans; the disclosed system may also alert the physicians to any outliers or variation in patients' symptoms.

The disclosed system may be trained by doctors and/or providers and/or patients to respond to specific clinical symptoms and exam findings in order to accurately document, diagnose and treat conditions while uploading to an expanding library data bank for storage and future retrieval and usage. The disclosed system will provide doctors and patients with an overriding capacity to document customized symptoms and clinical findings. Based on the pattern and frequency of usage by patients and doctors, the disclosed system is capable of learning habits.

A physician's documented clinical findings may be loaded into the disclosed system will automatically populate a series of potential diagnoses and treatment options to be selected. As a result, the physician's diagnostic and treatment selections, based on the frequency of specific clinical findings, will create the most mathematically probable diagnoses and treatments on an ongoing basis.

Thus, in accordance with disclosed embodiments, a Physician's clinical data inputs may train the disclosed software system to respond and recognize to the appropriate clinical scenarios for mathematically predictable accurate diagnoses and treatments. This is helpful in the process of determining common diagnosis with corresponding treatments along with clinical outliners and variations in a patient's conditions which are based on the utilization of mathematic probability that is reflective of the pattern and frequency of the specific diagnostic and treatment options selected.

Examples highlighting the technical capabilities of the disclosed software application are provided as follows:

1. Upon a physician's completion of a clinical exam input into the disclosed system, a series of potential diagnoses may be selected. For example, the potential diagnoses may appear on an electronic screen with corresponding treatment options allowing the physician to select the most accurate diagnosis and treatments available based on their in-person and hands-on clinical exam. In addition, the physician will be able to override the software's algorithm template diagnosis and treatment selections that may have been initially provided and, in-turn, create an alternative and/or more accurate diagnosis and treatment plan based on their knowledge, training, and clinical experience. In a disclosed embodiment, the disclosed input to create an alternative and/or more accurate diagnosis and treatment plan may include a voice dictated or type-texted input configured to the disclosed system. Such added information may be immediately uploaded to the clinical data bank of the disclosed software for permanent storage and later retrieval and usage by any participating provider connected to the disclosed system.

Accordingly, the disclosed software is configured to receive customized clinical data input from providers and patients, store the information in the data software bank and become available for ongoing future use by both patients and providers utilizing the disclosed software application. In accordance, with disclosed embodiments, machine learning of the disclosed system and software applications occurs over time, based on the pattern and frequency of use, the disclosed system continues to expand stored clinical information and mathematically produce accurate diagnoses and treatments based on the treating physician's clinical judgment.

2. The interactive 3D anatomical model initially utilized by a patient to visually illustrate their symptoms, may include a virtual avatar, for example, configured in a likeness of the user for a more personalized application feature. This interactive 3D model may also be analyzed by a treating physician prior to an in-person, hands-on exam; and by juxtapose analysis an exact mirror image of 3D model may be displayed for a provider's clinical exam documentation. The mirror-imaged 3D interactive model may allow a physician to utilize the disclosed software features to illustrate clinical findings which include, but are not limited to, medical diagnosis such as edematous swelling, hives, psoriasis, herpes, cuts, bruises and lacerations, etc.

In some embodiments, areas of the 3D anatomical model may be configured to be highlighted as an area of symptomatic emphasis by a patient and/or treatment doctor. For example, a patient may touch or select their specific sites of symptoms using the 3D interactive avatar model. That area of the 3D interactive avatar model is configured to immediately turn a different color. For example, an area of the 3D interactive avatar model may be configured to turn to the color red to illustrate bleeding, blue for cold or coolness of the skin and limbs, yellow or green for pus depicting drainage from a body part, black for fungus of nails or gangrenous skin from poor peripheral circulation, pink, red or yellow for eye conjunctiva depicting possible eye infections or jaundice.

The illustrative depictions of a provider's exam may be uploaded to the patient's electronic health record (EHR) and formatted into a comprehensive finalized medical report upon completion of the in-person, hands-on medical exam. Any selections may be confirmed by the patient provider's in-person, hands-on exam findings, for example, on a juxtaposed 3D avatar presented to the patient provider for a respective patient. On the illustrative side of the juxtaposed 3D interactive patient avatar presented to the patient provider, verifications and confirmations may be immediately uploaded to the EHR and subsequently the final medical note or comprehensive report directly and immediately following the provider's input into the disclosed system or software application. Thus, the patient provider will document their in-person, hands on clinical exam. Any digital illustrations of each clinical exam finding may be directly verified prior to input into the disclosed system for immediate uploading into the patient's EHR and final medical report.

3. Utilizing the disclosed digital medical algorithm templates for both patients and providers may override the disclosed software templates in order to give additional detailed information to describe a patient's clinical diagnosis. The disclosed system may include such options as having limited voice dictation and type-text features configured thereto. Further, the disclosed system may also accommodate dictated voice command as an option to the one touch responses in a disclosed digital software algorithm platform for both patients and doctors to document clinical information in the disclosed system for the purpose of completing a comprehensive medical report upon finalizing an in-person, hands-on medical exam.

4. Outpatient medical visits may also utilize features of the disclosed dual software application by allowing patients to input their clinical histories and symptoms into the disclosed system. Such clinical histories and symptoms may be uploaded to the EHR prior to arriving at a provider's office for examination. Such action may service as a part of a pre-registration procedure and begin the start of populating data to a received clinic remotely. When a provider reviews the patient's clinical symptoms, for example, on the patient's portal, the disclosed software may format a working clinical medical note which may be finalized upon the provider's clinical exam findings Immediately, at the end of the patient-provider in-person, hands-on clinical visit, the entire medical report may be finalized with increased accuracy, timelines and without further transcription.

5. Disclosed embodiments of the software application may include a capacity to connect with satellite modems for speed of transmission, security encryption and communication as well as all other traditional internet modems and communication systems.

The above-described embodiments of the invention are presented for purposes of illustration and not of limitation. While these embodiments of the invention have been described with reference to numerous specific details, one of ordinary skill in the art will recognize that the invention can be embodied in other specific forms without departing from the spirit of the invention. Thus, one of ordinary skill in the art would understand that the invention is not to be limited by the foregoing illustrative details, but rather is to be defined by the appended claims.

The system, as described in the present technique or any of its components, may be embodied in the form of a computer system. Typical examples of a computer system includes a general-purpose computer, a programmed micro-processor, a micro-controller, a peripheral integrated circuit element, and other devices or arrangements of devices that are capable of implementing the steps that constitute the method of the present technique.

The computer system comprises a computer, an input device, a display unit and/or the Internet. The computer further comprises a microprocessor. The microprocessor is connected to a communication bus. The computer also includes a memory. The memory may include Random Access Memory (RAM) and Read Only Memory (ROM). The computer system further comprises a storage device. The storage device can be a hard disk drive or a removable storage drive such as a floppy disk drive, optical disk drive, etc. The storage device can also be other similar means for loading computer programs or other instructions into the computer system. The computer system also includes a communication unit. The communication unit allows the computer to connect to other databases and the Internet through an I/0 interface. The communication unit allows the transfer as well as reception of data from other databases. The communication unit may include a modem, an Ethernet card, or any similar device which enables the computer system to connect to databases and networks such as LAN, MAN, WAN and the Internet. The computer system facilitates inputs from a user through input device, accessible to the system through I/0 interface.

The computer system executes a set of instructions that are stored in one or more storage elements, in order to process input data. The storage elements may also hold data or other information as desired. The storage element may be in the form of an information source or a physical memory element present in the processing machine.

The set of instructions may include various commands that instruct the processing machine to perform specific tasks such as the steps that constitute the method of the present technique. The set of instructions may be in the form of a software program. Further, the software may be in the form of a collection of separate programs, a program module with a larger program or a portion of a program module, as in the present technique. The software may also include modular programming in the form of object-oriented programming The processing of input data by the processing machine may be in response to user commands, results of previous processing or a request made by another processing machine.

While the present disclosure has been disclosed with references to certain embodiments, numerous modification, alterations, and changes to the described embodiments are possible without departing from the sphere and scope of the present disclosure, as defined in the appended claims. Accordingly, it is intended that the present disclosure not be limited to the described embodiments, but that it has the full scope defined by the language of the following claims, and equivalents thereof.

What is claimed is:

1. A process to self-report symptoms to a physician for an in-person examination, comprising:
transmitting a patient mobile application to a patient mobile device associated with a patient who has a medical issue and is seeking in-person examination and diagnosis of the medical issue to be treated, wherein when the patient mobile application is installed on the patient mobile device the patient is able to seek in-person examination and treatment from an available nearby physician in a pool of nearby physicians;
transmitting a physician mobile application to a physician mobile device associated with a physician who is available to examine patients nearby a physical location of the physician, wherein when the physician mobile application is installed on the physician mobile device, an identify and qualifications of the physician are verified, and upon receiving physician approval to receive nearby requests for appointments by patients, the physician is listed among a pool of physicians in an area that is nearby the physical location of the patient;
receiving, from the patient mobile application running on the patient mobile device, answers to a series of questions paired with one or more 3D human anatomical model(s) to gather initial patient data;
receiving, from the patient mobile application running on the patient mobile device, an opt-in request to locate a nearby physician and schedule an in-person appointment;
receiving, from the patient mobile application running on the patient mobile device, an approval to participate in and pay for an in-person examination according to the scheduling of the in-person appointment;
transmitting a request to schedule the in-person appointment to the physician mobile application running on the physician mobile device;
receiving an acceptance, from the physician mobile application running on the physician mobile device, to schedule the in-person appointment to examine the patient;
utilizing by the physician mobile application running on the physician mobile device, a data platform and predictive diagnoses to complete an in-person examination of the patient; and
prescribing a treatment plan for the patient based on the completed in-person examination of the patient by the physician.
